(19) United States
(12) Patent Application Publication
(10) Pub. No.: US 2024/0189383 A1
(43) Pub. Date: Jun. 13, 2024

(54) Application of Inhibitors Targeting CD226 Molecules in Anti-Tumor Metastasis

(71) Applicant: AIR FORCE MEDICAL UNIVERSITY, Xi'an, Shaanxi (CN)

(72) Inventors: Ran Zhuang, Xi'an, Shaanxi (CN); Yuan Zhang, Xi'an, Shaanxi (CN); Yong Ding, Xi'an, Shaanxi (CN); Yitian Liu, Xi'an, Shaanxi (CN)

(73) Assignee: AIR FORCE MEDICAL UNIVERSITY, Xi'an, Shaanxi (CN)

(21) Appl. No.: 18/554,465

(22) PCT Filed: Apr. 26, 2023
(86) PCT No.: PCT/CN2023/090750

§ 371 Date: Oct. 7, 2023

(30) Foreign Application Priority Data
Sep. 1, 2022 (CN) CN202211066187.6

(51) Int. Cl. A61K 38/08; A61K 31/343; A61K 31/7034; A61K 31/7048; A61K 38/04; A61P 35/04

(52) U.S. Cl. CPC A61K 38/085; A61K 31/343; A61K 31/7034; A61K 31/7048; A61K 38/043; A61K 38/08; A61P 35/04

(57) ABSTRACT

The present disclosure relates to the field of biotechnology, and in particular to an application of inhibitors targeting CD226 molecules in anti-tumor metastasis. In view of the important role of platelets in mediating tumor metastasis, the present disclosure develops small-molecule inhibitors with blocking effects by targeting CD226 molecules, an important target. It can destroy the interaction between platelets and tumor cells, and inhibit the metastasis of tumor cells, and it is important for research and development of new methods to control tumor metastasis.

### CROSS-REFERENCE TO RELATED APPLICATION

This application is a Section 371 National Stage Application of International Application No. PCT/CN2023/090750, filed on Apr. 26, 2023, entitled “Application of Inhibitors Targeting CD226 Molecules in Anti-Tumor Metastasis”, which claims priority to Chinese Application No. 202211066187.6, filed on Sep. 1, 2022, incorporated herein by reference in their entirety.

### TECHNICAL FIELD

The present disclosure relates to the field of biotechnology, and in particular to an application of inhibitors targeting CD226 molecules in anti-tumor metastasis.

### BACKGROUND

Platelets have a clear role in promoting the metastasis of tumor cells. The main mechanisms include: tumor cells promote platelet activation and aggregation, and the activated platelets cross-link with each other and protect tumor cells from the damage of cyclic shear stress and from being killed by NK cells; platelets promote tumor anoikis resistance, EMT, angiogenesis and extravasation through direct contact or release of soluble factors; platelets recruit a variety of immune cells to exert immunomodulatory effects and assist tumor metastasis. In this process, cell adhesion molecules play an important role in mediating the cross-linking between platelets and platelets and between platelets and tumor cells. The cell adhesion molecules can also initiate various pathophysiological functions of platelets and tumor cells through various signaling pathways.

Cell adhesion molecules (CAM) mediate for adhesion and communication between cells and cells and between cells and extracellular matrix (ECM). Cell adhesion molecules on platelets play the role of adhesion and signaling molecules in promoting tumor metastasis.

Tumor cell metastasis is a serious problem faced by most patients with malignant tumors. 90% of tumor-related deaths are caused by tumor cell metastasis rather than the primary tumor. Even after surgery, chemotherapy, targeted therapy and immunotherapy, there is still a huge risk of tumor cell metastasis, and metastasis is always a life-threatening danger to patients. At present, one of the important aspects in clinical treatment and basic tumor research is how to reduce the metastasis path of tumor cells.

The high platelet status of cancer patients promotes tumor growth, angiogenesis, metastasis and tumor-related thrombosis and thereby participates in every process of tumor development, becoming an independent adverse prognostic factor. Based on the involvement of platelets in tumor progression, numerous experimental models and results of epidemiological studies on the use of antiplatelet drugs to prevent tumors, platelets can be used as a potential target, and interfering with platelets can help reduce tumor metastasis and mortality. Therefore, platelets are of far-reaching significance and value in the study of tumor progression mechanisms and the development of anti-tumor treatments. More and more experiments are needed to confirm the clinical effect of platelet intervention in combination with other anti-tumor drugs.

The existing technologies used to solve this technical problem are mainly platelet inhibitors. The main inhibitors that have been studied in tumor treatment include: I. Cyclooxygenase inhibitors: for example, aspirin, which can block the conversion of arachidonic acid into TXA2 and inhibit platelet aggregation. A large meta analysis showed that aspirin not only reduces the risk of distant metastasis but also reduces the risk of death incurred by tumor. II. ADP P2Y12 receptor antagonists: for example, ticagrelor has been shown to have the ability to inhibit tumor adhesion and metastasis in mouse melanoma and breast cancer models. III. Platelet protease-activated receptor-1 inhibitor: it can block thrombin-mediated platelet activation and aggregation, knock out platelet protease-activated receptor-1 and reduce the invasive ability of melanoma cells.

However, the problems with the above technology lie in that: cyclooxygenase inhibitors, ADP P2Y12 receptor antagonists, and platelet protease-activated receptor-1 inhibitors have relatively large side effects, because these molecules are responsible for normal physiological functions of hemostasis and repair of vascular endothelial cells of platelets. When these drugs inhibit tumor metastasis, they destroy the normal physiological functions of platelets to a certain extent, causing obvious side effects. Therefore, there is a need to develop an anti-tumor metastasis site with less side effects.

### SUMMARY

In order to solve the above technical problems, the present disclosure provides an application of inhibitors targeting CD226 molecules in anti-tumor metastasis.

The purpose of the present disclosure is to provide an application of inhibitors targeting CD226 molecules in anti-tumor metastasis.

According to embodiments of the present disclosure, the CD226 molecules are located on platelets

According to embodiments of the present disclosure, interfering with CD226 molecules on the platelets is capable of reducing platelet activation and inhibiting tumor metastasis.

According to embodiments of the present disclosure, the CD226 molecules serve as a site which regulates interaction of the platelets and tumor cells, and the inhibitors for inhibiting tumor metastasis are prepared accordingly.

According to embodiments of the present disclosure, the inhibitors are small-molecule inhibitors or macromolecule inhibitors.

According to embodiments of the present disclosure, the small-molecule inhibitors are angiotensin III, neohesperidin, [Leu5]-enkephalin, epimedin B, methylhesperidin, salvianolic acid B, bradykinin (2-9), echinacoside, astragaloside III or poliumoside.

According to embodiments of the present disclosure, the tumor is mouse osteosarcoma cell line K7M2 or mouse melanoma cell line B16F10.

According to embodiments of the present disclosure, the CD226 molecules serve as a site which regulates interaction of the platelets and tumor cells, and a tumor detection kit is prepared accordingly.

Compared with the prior art, the present disclosure has the following beneficial effects:

It has been found by studies that CD226 has a low impact on the normal physiological functions of platelets. It has been found by experiments that targeted inhibitors have a slight impact on functions such as hemostasis and have few side effects. However, they have a good inhibitory effect on tumor metastasis and therefore it can serve as a good therapeutic target for anti-tumor metastases.

In view of the important role of platelets in mediating tumor metastasis, small-molecule inhibitors with blocking effects are developed by targeting the CD226 molecules, an important target, the inhibitors are expected to destroy the interaction between platelets and tumor cells, and inhibit the metastasis of tumor cells, and it is important for research and development of new methods to control tumor metastasis.

It should be noted that the anti-tumor mechanism of platelet CD226 is different from that of the CD226 expressed in other cells; for the CD226 molecules expressed in T cells and NK cells, they mainly act as activating receptors to activate T cells and NK cells to directly kill tumor cells; while in the present disclosure, the CD226 has different effects on platelets. This difference is due to the different physiological functions of platelets and T cells/NK cells. The main discovery of the present invention is that blocking the function of CD226 on platelets can block the promoting role of platelets in tumor metastasis.

The present disclosure discovered and verified for the first time the blocking effect of 10 small-molecule compounds on CD226.

### BRIEF DESCRIPTION OF THE DRAWINGS

FIG. 1 is the technical route of the present disclosure.

FIG. 2 shows the results of platelet aggregation experiment;
From left to right, thrombin induced WT platelets, tumor cell induced WT platelets, and tumor cells are used to induce CD226KO (hereinafter referred to as KO) platelets.

FIG. 3 shows the results of detecting adhesion of platelets induced by tumor cells using fluorescent probes;
A shows the results of WT and KO induction of tumor cells under a fluorescence microscope, and B shows the statistical results of fluorescence intensity detected by a microplate reader (n=4), the length of the ruler is 275 μm.

FIG. 4 shows the results of flow cytometry to detect tumor cell induced platelet activation;
A is the density maps of tumor cell induced WT and KO platelet activation, and B is the statistical result of the proportion of platelet activation marker CD62P (n=3).

FIG. 5 shows the general results of metastatic lesions in vivo experiments on tumor metastasis;
A shows the general results of metastatic lesions in the lower lobe of the right lung of CD226^{f1/f1} mouse and CD226^{f1/f1}PF4-Cre mouse, and B shows the counting statistical result of metastatic lesions in the whole lung (n=4), the length of the ruler is 75 μm.

FIG. 6 shows the results of HE staining of metastatic lesions under the microscope in the in vivo experiment of tumor metastasis;
A shows the results of HE staining of metastatic lesions in CD226^{f1/f1} mouse and CD226^{f1/f1}PF4-Cre mouse under the microscope, and B shows the counting statistical result of metastatic lesions under the microscope (n=4).

FIG. 7 shows the computer molecular docking scoring results of 10 candidate inhibitors.

FIG. 8 shows the Chinese names of 10 candidate inhibitor compounds.

FIG. 9 is a schematic diagram of poliumoside 3D (A) and 2D (B) docking interaction.

FIG. 10 shows the results of detecting inhibitory efficiency of candidate inhibitors using fluorescent probes;
A is the statistical results of the inhibitory efficiency of 10 candidate inhibitors, and B is the inhibitory effect diagram of salvianolic acid B (Sal B) under a fluorescence microscope (n=4), the length of the ruler is 275 μm.

FIG. 11 shows the statistical results of inhibitory efficiency of candidate inhibitors detected by flow cytometry (n=3).

FIG. 12 shows the results of density chart of inhibitory efficiency of candidate inhibitors detected by flow cytometry.

### DETAILED DESCRIPTION OF EMBODIMENTS

In order to enable those skilled in the art to better understand and implement the technical solution of the present disclosure, the present disclosure will be further described below with reference to specific embodiments and drawings.

In the description of the present disclosure, unless otherwise specified, all reagents used are commercially available, and all methods used are common techniques in this field.

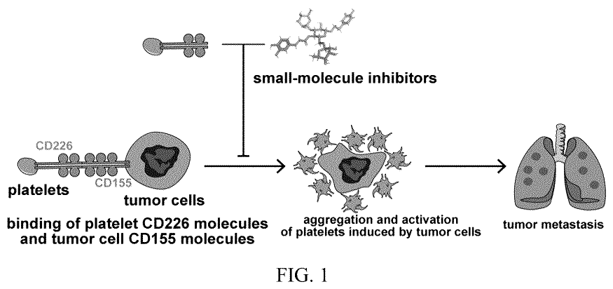

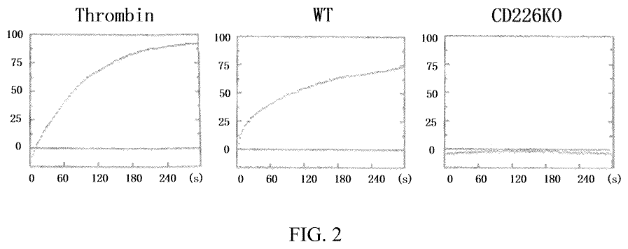

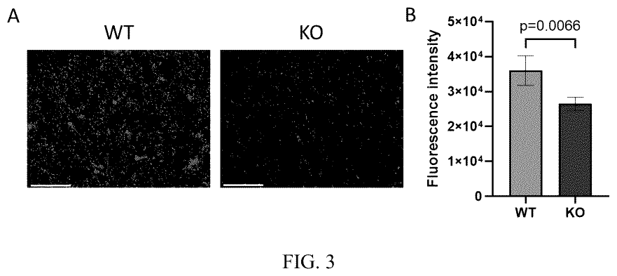

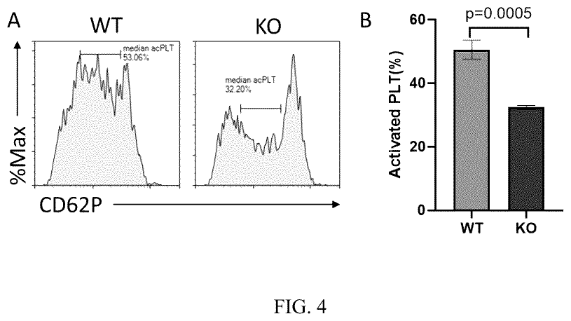

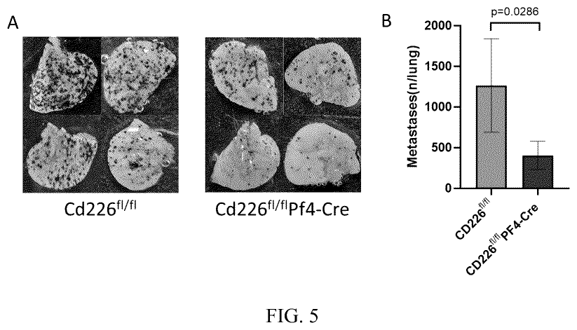

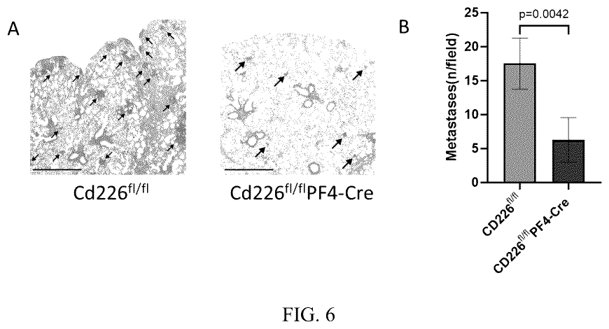

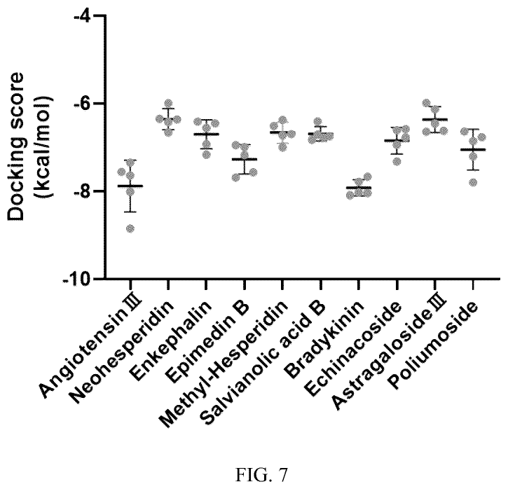

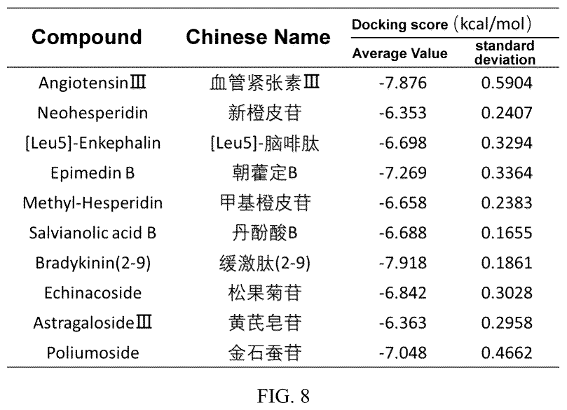

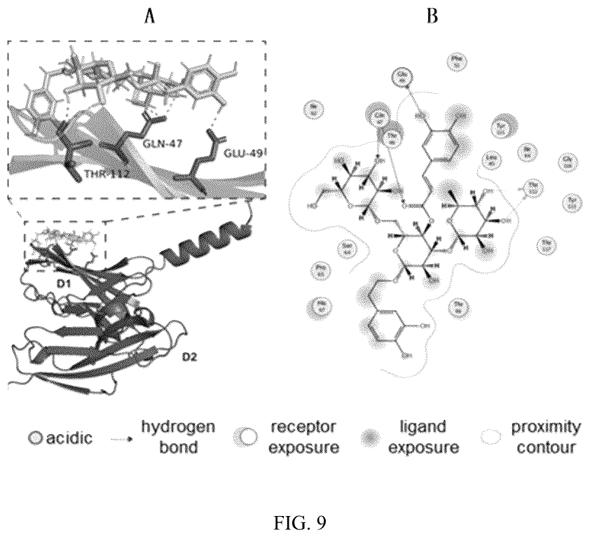

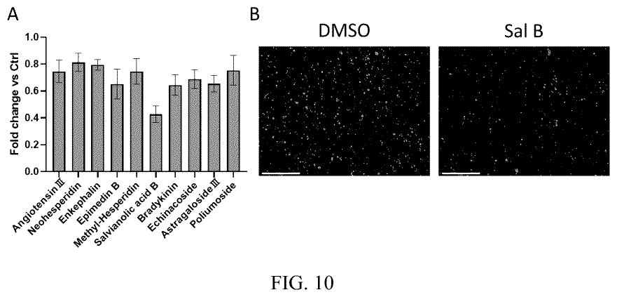

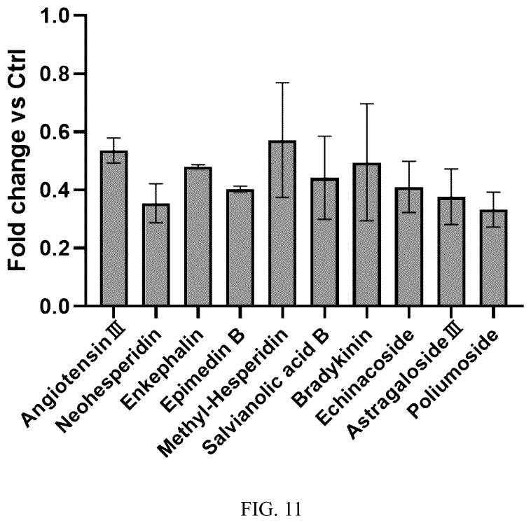

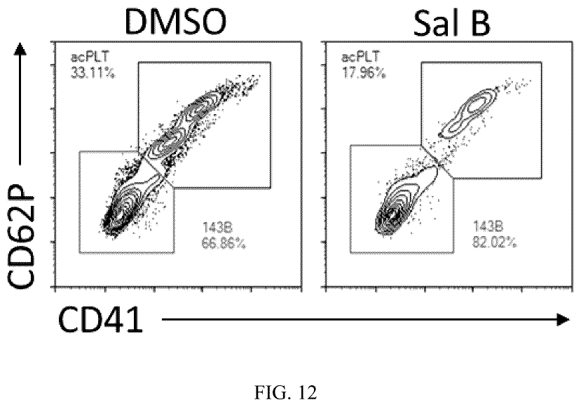

The technical route of the present disclosure is shown in FIG. 1.

I. Tumor Cellinduced Platelet Aggregation and Activation (TCIPA) detection

CD155-positive tumor cell lines were cultured, including the mouse-derived cell line mouse osteosarcoma cell line K7M2 (derived from BALB/c) and mouse melanoma cell line B16F10 (derived from C57BL/6J).

Preparation of mouse platelets: Anticoagulated mouse whole blood and Tyrode's buffer (137 mM NaCl, 2 mM KCl, 12 mM NaHCO₃, 0.3 mM NaH₂PO₄, 5.5 mM glucose, 5 mM HEPES, pH 7.3, 0.35% BSA, the solvent is ultrapure water) are mixed well by 1:1 (volume ratio), and centrifuged at 180×g for 10 minutes at room temperature. The supernatant is platelet rich plasma (PRP); the PRP is transferred to a new centrifuge tube, and centrifuged at 2000 rpm for 10 minutes at room temperature. The sediment is platelets. Then appropriate volume of Tyrode's buffer is added and resuspended and then it can be used immediately.

Platelet aggregation experiment: 250 μl platelets (1.5×10⁸/ml) are added to a test cup of a platelet aggregator (LBY-NJ4). In the experimental group, 4 μl tumor cell suspension (4×10⁵/ml) is added for induction aggregation after 30 sec. Thrombin (1 U/ml) is used as a positive control. The test results are shown in FIG. 2, from left to right, thrombin is used to induce WT (wild-type) platelets, tumor cells are used to induce WT (wild-type) platelets, and tumor cells are used to induce CD226KO (CD226 knockout) platelets. The results show that the ability of tumor cells to induce WT platelet aggregation is close to that of thrombin. However, after knocking out the CD226 molecules in platelets, tumor cells are almost unable to induce their aggregation, indicating that CD226 molecules are involved in tumor cell induced platelet aggregation.

Detection of adhesion of platelets induced by tumor cells using fluorescent probe: after tumor cell dissociation, seed them in a 96-well plate (5×10⁵/ml) at 100 μl per well, and culture them overnight in a 37° ° C. incubator. Take the washed platelets (1.5×10⁸/ml), add DIL fluorescent probes, and stain in a water bath at 37° C. for 5 minutes in the dark, wash off the excess probe, add 100 μl of platelets per well to a 96-well plate and incubate in an incubator at 37° C. for 30 minutes, wash away non-adherent platelets, fix in 4% paraformaldehyde in the dark for 10 minutes, put it into a microplate reader (540 nm-570 nm) to detect the fluorescence intensity, and take pictures under the RFP channel of a fluorescence microscope. The test results are shown in FIG. 3. A is the adhesion results of WT and KO induction of tumor cells under a fluorescence microscope, and B is the statistical results of fluorescence intensity detected by a microplate reader (n=4). The results show that the ability of platelets to adhere to the surface of tumor cells is significantly reduced after knocking out the CD226 molecules.

Flow cytometry to detect platelet activation induced by tumor cells: first, prepare a mouse platelet suspension (1.5×10⁸/ml), dissociate the tumor cells with trypsin and then resuspend them in a culture medium containing 10% (volume ratio) fetal bovine serum (5×10⁵/ml). Mix 300 μl of platelet suspension and 100 μl of tumor cell resuspension, and incubate them in a fine incubator at 37° C. for 30 min, detect the level of P-selectin (CD62P; clone number: Psel.KO2.3) and αIIbβ3 (CD41; clone number: JON/A) and other platelet surface activation markers by flow cytometry. The test results are shown in FIG. 4, A is the density maps of tumor cell induced WT and KO platelet activation, and B is the statistical result of the proportion of platelet activation marker CD62P (n=3). The results show that although there was no difference in the platelets with the strongest CD62P fluorescence intensity after knocking out CD226, the activation ability of most platelets is significantly weakened.

Combining the results of the three parts of experiments, it shows that after knocking out the CD226 molecules in platelets, the ability of tumors to induce platelet aggregation and activation is weakened.

### II. In Vivo Experiments on Tumor Metastasis

Cultivate melanoma B16F10 cells, and inject them into a mouse through the tail vein (1.0×10⁶/mouse). After 10 days, the mouse is euthanized and the lung tissue is collected, the right lower lobe of the mouse is taken for gross observation of the pathological tissue. The left lower lobe of the lung was taken for HE section microscopy to observe and count the number of tumor cell metastatic lesions.

The in vivo experimental results are shown in FIGS. 5 and 6. The results show that both grossly and microscopically, the lung tumor metastatic lesions of mouse with platelet-specific CD226 knockout are significantly less than those of the control mouse (n=4), indicating that platelet CD226 molecules can promote tumor lung metastasis.

### III. CD226 Inhibitor Screening

Computer molecular docking simulation: Computer virtual screening is performed on the binding pocket of CD226-CD55, hoping to obtain small-molecule compounds with strong binding force to the target protein CD226. Download the crystal structure of CD226 (PDB ID: 6O3O) from the RCSB PDB database, use the Protein Preparation Wizard module of Schrödinger Maestro 11.4 software to optimize hydrogenation of CD226 protein, delete water molecules, and repair missing residues, side chains, etc. This is followed by energy optimization (OPLS2005 force field, RMSD of 0.30 Å). Use the Receptor Grid Generation module to create a grid file for the processed protein, and generate a grid file based on the CD226 binding pocket (the key amino acid residues in the interface are THR46/GLN47/GLU49/SER64/HIS67/VAL70/AGR72/TYR113 /PRO114/GLY116/THR117). The 2D formats of Life Chemicals 50K Diversity Library (containing 50.2K compounds) and MCE Bioactive Compound Library Plus (containing 12.6K compounds) are exported to 3D structures through the Lig Prep Module of Schrödinger software, and the Virtual Screening Workflow module is used for virtual screening. Use the Glide module for molecular docking, first, use high-throughput virtual screening (HTVS) mode to screen small-molecule compounds, select the top 10% of the compounds with scoring values and use the standard precision (SP) mode for the second round of screening; then select the top 10% of the compounds with scoring values, and use the extra precision (XP) mode to perform the third round of screening to obtain the ranking of the small-molecule compounds. Manually review the binding ability of the target and the compound, the compound structure, etc., and finally select 10 compounds that are economical, easy to obtain, and have known mild side effects from the top 200 compounds as candidate inhibitors. Use the Dock option of the Compute module of the MOE software to calculate the Docking scores of the 10 candidate inhibitors, and output the top five scores and 2D interaction diagrams for each candidate inhibitor. Use Pymol to draw a 3D interaction diagram. The screening results are shown in FIG. 7, FIG. 8 and FIG. 9. FIG. 7 shows the computer molecular docking scoring results of 10 candidate inhibitors; FIG. 8 shows the Chinese names of the compounds, the docking scores of the 10 compounds are all below −5 points, indicating that they can well bind to the binding pocket of the CD226 molecules in terms of spatial structure. docking. FIG. 9 is a schematic diagram of poliumoside 3D (A) and 2D (B) docking interaction, it forms multiple hydrogen bonds with the CD226 molecules and can fit the binding pocket well.

Detection of inhibitory efficiency of candidate inhibitors using fluorescent probes: add candidate inhibitors (25 μg/ml) after platelet staining, and add an equal volume of DMSO or double-distilled water to the control group according to the different solvents of the drug storage solution. The remaining steps are the same as the first part of the detection of adhesion of platelets induced by tumor cells using fluorescent probe. The inhibition efficiency is expressed by fold change (FC), the ratio of the fluorescence intensity of the experimental group to the corresponding control group after subtracting the background fluorescence intensity of all samples. The screening results are shown in FIG. 10. A is the statistical results of the inhibitory efficiency of 10 candidate inhibitors, and B is the inhibitory effect diagram of salvianolic acid B (Sal B) under a fluorescence microscope. The results show that all 10 candidate inhibitors could significantly inhibit the adhesion between tumor cells and platelets.

Flow cytometry to detect the inhibitory efficiency of candidate inhibitors: add candidate inhibitors (25 μg/ml) or DMSO, double-distilled water to the platelets before mixing them with the tumor cell suspension. The remaining steps are the same as the first part of flow cytometry to detect platelet activation induced by tumor cells. The inhibitory efficiency is expressed by fold change, that is, the ratio of CD41-positive events in the experimental group to the control group in the event of tumor cell size. The screening results are shown in FIG. 11 and FIG. 12. FIG. 11 shows the statistical results of inhibitory efficiency of candidate inhibitors detected by flow cytometry. FIG. 12 shows the results of density chart of inhibitory efficiency of candidate inhibitors detected by flow cytometry.

In summary, the research results of the present disclosure show that: I. Platelets play an important role in promoting tumor cell metastasis; II. CD226 is expressed at a high level on platelets; III. Intervening with CD226 molecules on platelets can effectively reduce platelet activation and inhibit tumor metastasis. Therefore, the CD226 molecules serve as a site which regulates interaction of the platelets and tumor cells, and small-molecule inhibitors that can effectively inhibit tumor metastasis have been selected, and it provides experimental evidence for the development of drugs to prevent and treat tumor metastasis.

It should be noted that, when the present disclosure involves a numerical range, it should be understood that the two endpoints of each numerical range and any numerical value between the two endpoints can be selected. Since the steps and methods used are the same as those in the embodiment, in order to avoid redundancy, the present disclosure describes preferred embodiments. Although the preferred embodiments of the present disclosure have been described, additional changes and modifications may be made to these embodiments by those skilled in the art once the basic inventive concepts are apparent. Therefore, it is intended that the appended claims should be construed to include the preferred embodiments and all changes and modifications that fall within the scope of the present disclosure.

Obviously, those skilled in the art can make various changes and modifications to the present disclosure without departing from the spirit and scope of the present disclosure. In this way, if these changes and modifications of the present disclosure fall within the scope of the claims of the present disclosure and equivalent technologies thereof, the present disclosure is also intended to include these changes and modifications.

What is claimed is:

1. An application of inhibitors targeting CD226 molecules in anti-tumor metastasis.

2. The application of inhibitors targeting CD226 molecules in anti-tumor metastasis according to claim 1, wherein the CD226 molecules are located on platelets

3. The application of inhibitors targeting CD226 molecules in anti-tumor metastasis according to claim 2, wherein interfering with CD226 molecules on the platelets is capable of reducing platelet activation and inhibiting tumor metastasis.

4. The application of inhibitors targeting CD226 molecules in anti-tumor metastasis according to claim 2, wherein the CD226 molecules serve as a site which regulates interaction of the platelets and tumor cells, and the inhibitors for inhibiting tumor metastasis are prepared accordingly.

5. The application of inhibitors targeting CD226 molecules in anti-tumor metastasis according to claim 4, wherein the inhibitors are small-molecule inhibitors or macromolecule inhibitors.

6. The application of inhibitors targeting CD226 molecules in anti-tumor metastasis according to claim 4, wherein the small-molecule inhibitor is angiotensin III, neohesperidin, [Leu5]-enkephalin, epimedin B, methylhesperidin, salvianolic acid B, bradykinin (2-9), echinacoside, astragaloside III or poliumoside.

7. The application of inhibitors targeting CD226 molecules in anti-tumor metastasis according to claim 1, wherein the tumor is mouse osteosarcoma cell line K7M2 or mouse melanoma cell line B16F10.

8. The application of inhibitors targeting CD226 molecules in anti-tumor metastasis according to claim 2, wherein the CD226 molecules serve as a site which regulates interaction of the platelets and tumor cells, and a tumor detection kit is prepared accordingly.

9. The application of inhibitors targeting CD226 molecules in anti-tumor metastasis according to claim 2, wherein the tumor is mouse osteosarcoma cell line K7M2 or mouse melanoma cell line B16F10.

10. The application of inhibitors targeting CD226 molecules in anti-tumor metastasis according to claim 3, wherein the tumor is mouse osteosarcoma cell line K7M2 or mouse melanoma cell line B16F10.

11. The application of inhibitors targeting CD226 molecules in anti-tumor metastasis according to claim 4, wherein the tumor is mouse osteosarcoma cell line K7M2 or mouse melanoma cell line B16F10.

12. The application of inhibitors targeting CD226 molecules in anti-tumor metastasis according to claim 5, wherein the tumor is mouse osteosarcoma cell line K7M2 or mouse melanoma cell line B16F10.

13. The application of inhibitors targeting CD226 molecules in anti-tumor metastasis according to claim 6, wherein the tumor is mouse osteosarcoma cell line K7M2 or mouse melanoma cell line B16F10.
